(19) United States
(12) Patent Application Publication
(10) Pub. No.: US 2002/0132875 A1
(43) Pub. Date: Sep. 19, 2002

(54) Solid nanocomposites and their use in dental applications

(75) Inventor: Lisa Stadtmueller, Wheaton, IL (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Dental Technologies, Inc.

(21) Appl. No.: 10/034,807

(22) Filed: Dec. 28, 2001

Related U.S. Application Data
(60) Provisional application No. 60/259,045, filed on Dec. 29, 2000.

(51) Int. Cl. C08K 3/34; A61F 2/00

(52) U.S. Cl. 523/115; 524/445

(57) ABSTRACT

The present invention provides for the composition, method of preparing and method of using a nanocomposite in dental applications. The use of the nanocomposite in dental applications substantially influences the dental products strength, durability, longevity, barrier properties and other desirable physical characteristics.

### BACKGROUND OF THE INVENTION

This application claims the benefit of U.S. Provisional patent application Ser. No. 60/259,045, filed on Dec. 29, 2000.

### FIELD OF THE INVENTION

This invention relates to the use of a nanocomposites in dental applications. This invention further relates to a method of substantially increasing the performance of a dental product by substantially influencing the materials strength, durability, longevity, barrier properties and other desirable physical characteristics.

1. Description of the Related Art

Nanocomposites are known in the art as a class of materials which exhibit ultrafine phase dimensions. State-of-the art nanotechnology provides a revolutionary industrial adaptation for improving the physical and mechanical properties of manufactured composites. These materials have generally shown that virtually all types and classes of nanocomposites lead to new and improved properties such as increased stiffness, strength, heat resistance, decreased moisture absorption and permeability.

Specifically, within the field of polymer nanotechnology, a novel approach to nanocomposite development has emerged. Researchers have enhanced properties and extended their utility. This greatly improved polymer nanocomposite containing layered-structured inorganic nanoparticles is generally referred to as polymer-silicate layered nanocomposites (PSLN). (See U.S. Pat. No. 6,136,908; U.S. Pat. No. 6,057,035; and U.S. Pat. No. 5,840,796). Currently, PSLN technology has been used in PET beverage containers (U.S. Pat. Nos. 5,876,812 and 5,972,448) and nylon composite automotive assemblies.

The current art of dental products suffers from many problems. The current dental composites have inadequate longevity, strength, and durability. This has especially been seen in the dental composite products. Dental composites are used as a dental restorative material and are classified by the FDA as a medical device. This often methacrylate-based composite is used to reconstruct damaged tooth structures, in situations where much of the natural tooth structure has been lost or damaged. Thus, dental composites are crucial for increasing tooth strength, durability, longevity, integrity and crown retention. The polymerized solid composite material must be able to withstand high mastication forces, temperature extremes and other external stresses in order to be retained in the mouth.

Ideally, the restorative composite material should last the life span of the patient. Current composite materials fall short of this goal. The reported failure rate is greater than 10% over a 5 year period. Material fatigue is influenced by filler size and shape, composition, texture, surface chemistry and several environmental factors, including humidity, pH and temperature. Many restorative composite materials fail because they are unable to function under moist conditions, withstand large temperature fluctuations and be subjected to repetitive load cycles. By conservative estimation, human dentition experiences over 1 million cycles of load every three years. This leads to fatigue failures in composites, which often occur via small fissures and propagate through the material during repeated loading. Durability of the materials is also effected by the nonuniform or excessive distributions of occlusal forces. Patients with bruxism or clenching habits (currently in excess of 15% of the population) place tremendous forces, often exceeding structural capacities, on conventional dental restoratives. The treacherous conditions of the mouth combined with the personal habits of a diverse population presents numerous opportunities for developing improved materials which more closely parallel the physical properties of natural teeth in order to increase the longevity of these important materials.

The coefficient of expansion of the composite material must closely approximate that of the natural tooth to ensure material retention during temperature fluctuations. The hydrolytic stability of the material is another critical factor and is accomplished through the minimization of water absorption. Increasing the depth of cure by light penetration is beneficial for light cure composites. This allows the dentist to build larger layers.

The imperfect but widely used material, amalgam, has been the choice for restorative composite material over the years because of its proven durability. Amalgam is the strongest synthetic material and is higher in compressive strength than dentin and enamel. However, restorative composite material is increasingly used in place of amalgam primarily because of amalgam's poisonous nature and potential health risks associated with mercury released from amalgam. In addition, composite materials are cosmetically more appealing because they can be colored to match the tooth shade and are more easily concealed under a crown than the dark metallic amalgam. Other prior art materials used are gold and ceramic materials. Gold has excellent mechanical properties; however, it is very expensive and frequently not acceptable for esthetic reasons. Ceramic materials are used due to their good appearance and their high abrasion resistance. However, they are liable to fracture and are difficult to process. See U.S. Pat. No. 6,114,409.

Restorative composite materials used by dentists are in dire need of strength improvements as well as durability and longevity. The prior art examples attempted to address the problems by mitigating shrinkage due to polymerization. For example, U.S. Pat. No. 5,955,514 teaches restorative adhesive dental materials, using a method of polymerization yielding a pliable polymerizable composition. Another example is U.S. Pat. No. 5,876,210 which teaches a dental polymer product and the process for preparing the composition. A further example, U.S. Pat. No. 5,061,184 teaches an adhesive composition for biomaterial use that has an excellent adhesive strength. Yet, a further example, U.S. Pat. No. 6,022,940, teaches a polymeric composition and composites prepared from spiroorthocarbonates and epoxy monomer.

The prior art also attempts to address the problem of strength and stiffness through fiber-reinforced composites as described in U.S. Pat. Nos. 6,069,192, 6,103,779 and 4,717,341. The fiber-reinforced composites contain an amorphous/non-crystalline acrylic resin thickener, the nature which permits fiber reinforcement to be easily incorporated. The composition can be molded using low pressure molding techniques and conditions to form dental appliances such as dental crowns and fixed and removable dental bridgework.

However, the prior art fails to address the need for improvements in the dental industry. Thus, there is a need in the art for the use of nanocomposites in dental applications to overcome the current disadvantages of dental materials. Specifically, a technology is needed that offers improvements to material strength, longevity, margin integrity and durability in both restorative composite materials including sealants, core materials, adhesives, bonding agents, veneering materials, cements, dentures, inlays, microfill composites, flowable composites, compomers, anterior composites, posterior composites, resin modified glass ionomers, condensable composites, all of which can be light cured, self cured or combination thereof and for use in tooth restorations, dental appliances, orthodontic appliances, bite plate appliances, denture base resins, temporary and permanent crowns and bridges and the like. As well, the use of nanocomposites can be used to overcome disadvantages in the medical industry such as orthopedic appliances, acrylic prosthesis, bone cements, adhesives and the like.

### SUMMARY OF THE INVENTION

This invention provides for the composition and method of using a nanocomposite in dental applications.

An objective of the present invention is to dramatically improve dental products properties by substantially influencing the materials strength, durability, longevity, barrier properties and other desirable physical characteristics.

Another objective of the present invention is to provide nanocomposite technology as a new medium for achieving even stronger composites by creating the ability to control chemical compounds and physical structures at the nanoscale. Nanometer sizes range from 1 to 100 nm, which is the range where phenomena associated with atomic and molecular interactions strongly influence the macroscopic properties of the material. When predicting the strength of composite material, one must consider if the specification of an internal stress (or strain) field is consistent with the external field imposed on the macroscopic body. These internal fields are locally influenced by the properties of the components: the size, the geometry, the connectivity of the filler and the relative dispersion of the distinct phase regions. The nanocomposite technology addresses each of the factors responsible for imparting strength of composite materials.

A further objective of the present invention is to provide significant improvements to composite strength by reducing the size of the filler particle. Strength enhancement in nanofilled composites arises from the interactions of its phases at the interfaces. By contrast, in a conventional composite based on micrometer-sized fillers the interfaces between the filler and the matrix constitute a much smaller volume fraction of the bulk material and therefore influence its properties to a much smaller extent. Nanocomposite technology enables the incorporation of chemically modified nano-sized particles into the composite material. When fully exfoliated, the nanocomposite particles are 1 nm (a billionth of a meter) across. These smaller filler particles have an amazingly high volume fraction of filler to resin interface, which greatly enhances material strength and provides greater possibilities for reduced shrinkage during polymerization. The increase volume fraction of the nanophase interfaces induces many new physical properties that are superior to current composite materials currently used in dental applications.

Yet, another objective of the present invention is to provide a composite with a more efficient geometry of the filler. The geometry of the filler is defined by its aspect ratio, which is the surface-to-width ratio of the particle. The aspect ratio determines the efficiency of the load transfer from the matrix to the fiber. The larger the aspect ratio, the more efficient the load transfer. Montmorillonite clay, one of the silicates used in resin-silicate layered nanocomposite, is a 2-to-1 layered smectite clay with a platelike structure. Each platelet is approximately 1 nm wide with surface dimensions of 100-1000 nm. This is considered to be an unusually high aspect ratio comparable only to those found for fiber-reinforced polymer composite.

A further objective of the present invention is to provide composite material that provides connectivity between the filler particle and the resin matrix. The connectivity of the filler to the resin matrix affects the ability of the composite to efficiently transfer load. In the nanocomposite technology, connectivity can be brought about via two different mechanisms, a bifunctional surface treatment and a silane-coupling agent. The bifunctional surface treatment can be polymerized with the resin, which tethers the filler to the resin matrix. The silicates can also be silane treated, which covalently links the edges of the filler to the resin. Thus, the nanocomposite technology offers an advantage over uncrosslinked filler composites with two distinct methods of linkage.

A further objective of the present invention is to provide unique ability to evenly disperse nano-sized silicate filler throughout the resin complex. Even dispersion is a problem in current composite materials. If the filler is not homogeneously dispersed, the optimum physical properties cannot be achieved. For example, if the resin matrix does not fully encapsulate the filler, voids are created. These voids weaken the material and propagate fractures. Also, if large agglomerates of small fillers are not broken down, any advantages achieved on the nano-scale are minimized. In the nanocomposite technology, the dispersion of the silicate platelet is accomplished via the surface modifier. The surface modifier is ion exchanged into closely layered silicate platelets. Originally, these filler particles are agglomerated and form layer stacks with each layer approximately 3.5 Å apart. The surface modifiers contain long carbon chains from the range of 8-20 carbons. These surface modifiers physically separate the layered silicate platelets at the molecular level upon absorption into the gallery spacing between each layer. The surface modifiers reduce the platelet-to-platelet attraction, promoting an expansion between each layer of the silicate platelets to a distance greater than 20 Å. The resin matrix is then fully intercalated between each layer. The polymerized exfoliated nanocomposite can then be separated in a continuous resin matrix by average distances of 180 Å or greater depending upon filler loading.

Improvements and modifications of nanocomposite technology will match or exceed several strength properties of the current prior art materials. Improving the materials strength, durability, longevity, barrier properties and other desirable physical characteristic would permit the composite to withstand the treacherous conditions of the mouth and consequently would positively impact oral health.

### BRIEF DESCRIPTION OF THE DRAWINGS

FIG. 1 is a schematic illustration depicting the action of the surface modifiers, which spread apart the gallery regions of the layered silicate platelets.

FIG. 2 is a schematic illustration depicting the exfoliation of the silicate platelets into the continuous resin matrix. Ideal exfoliation exposes the long chain functional groups of the surface modifier, causing greater accessibility of the functional group with the resin.

### DETAILED DESCRIPTION OF THE INVENTION

The use of a nanocomposite in dental applications provides for superior strength, durability, longevity, barrier properties and other desirable physical characteristics. One embodiment of the present invention is the method of using a solid nanocomposite in dental applications. Examples of such dental applications include but are not limited to, restorative composite materials such as, sealants, core materials, adhesives, bonding agents, veneering materials, cements, dentures, inlays, microfill composites, flowable composites, compomers, anterior composites, posterior composites, resin modified glass ionomers, condensable composites, all of which can be light cured, self cured or combination thereof, as well as dental appliances, orthodontic devices and appliances, bite plate appliances, denture base resins, temporary and permanent crowns and bridges and the like.

In a preferred embodiment, the invention provides a nanocomposite for use in dental applications in which the nanocomposite comprises a plurality of silicate platelets, one or more gallery regions spacing the plurality of silicate platelets from each other, at least one surface modifier ion-exchanged to each of the plurality of silicate platelets and a dentally compatible resin absorbed into the regions spacing the plurality of silicate platelets, the platelets and resin forming an intercalated and exfoliated structure.

In a preferred embodiment of the invention, silicate platelets are selected from the group consisting of smectite clay, vermiculite, halloysite, a mixed layered clay, a mica or sericite. Preferably, smectite clays such as montmorillonite, laponite, saponite, beidellite, nontronite, hectorite, swellable mica based mineral, stevensite or any synthetic analog thereof are employed as layered silicate platelets. Most preferably, the smectite clays used are montmorillonite and laponite. The montmorillonite is a naturally mined magnesium aluminum silicate clay with an enormously high aspect ratio of 1000-2000:1. Laponite clay is a synthetic clay with a higher purity than natural clays, yet it has a lower aspect ratio of 250:1. Preferably, montmorillonite and laponite exist in nano-sized aluminosilicate platelets. These platelets agglomerate into larger groupings of clay micels due to the surface attractions of silicate and oxygen tetrahedral or octahedral.

These clay minerals have layered lattice structures consisting of two-dimensional oxyanions separated by layers of hydrated cations. Various isomorphous substitutions by di- and trivalent cations result in negatively charged nanolayers (also referred to as “silicate layers or platelets”). The thickness of these layers are 0.92 nm. The nanolayers contain hydrated cations such as, for example, alkali or alkaline earth metal ions in the gallery regions (regions separating silicate layers; also referred to as galleries). Preferred hydrated cations are calcium and sodium ions. The negative charge of the layers is balanced by the hydrated cations within the gallery regions.

In a more preferred embodiment, the hydrated cations are exchanged with organic cations. These organic cations act as surface modifiers of the silicate platelet layers throughout the resin complex, thereby providing a mechanism for achieving optimum dispersion. Preferably, the organic cations used as surface modifiers include, but are not limited to, Bis(2-Hydroxyethyl) methyl tallow quaternary ammonium ion, dimethyl-2-ethyl hexyl hydrogenated tallow quaternary ammonium ion, methyl dihydroxyethyl hydrogenated tallow ammonium, aminododecanoic acid, polyoxyethylene decyloxypropylamine, and octadecyl trimethyl amine. The most preferred compounds are quaternary ammonium ions, which can be exchanged. The molecule must contain a minimum length of 8-20 carbons, to separate the layers effectively. Preferably, the molecule contains a length of 12-18 carbons. Each onium ion, which is ion exchanged to a layer, may contain a functional group that (1) matches the polarity of the resin to increase the absorbency of the resin into the gallery, and/or (2) contains a polymerizable group, which becomes bonded to the resin during polymerization. The first option allows the monomer to be fully intercalated. The second option in addition to full intercalation allows the resin to be chemically bonded to the surface modifier. Surface modifiers which contain unsaturated tallow are able to be polymerized by free radical polymerization to the methacrylate based resin matrix.

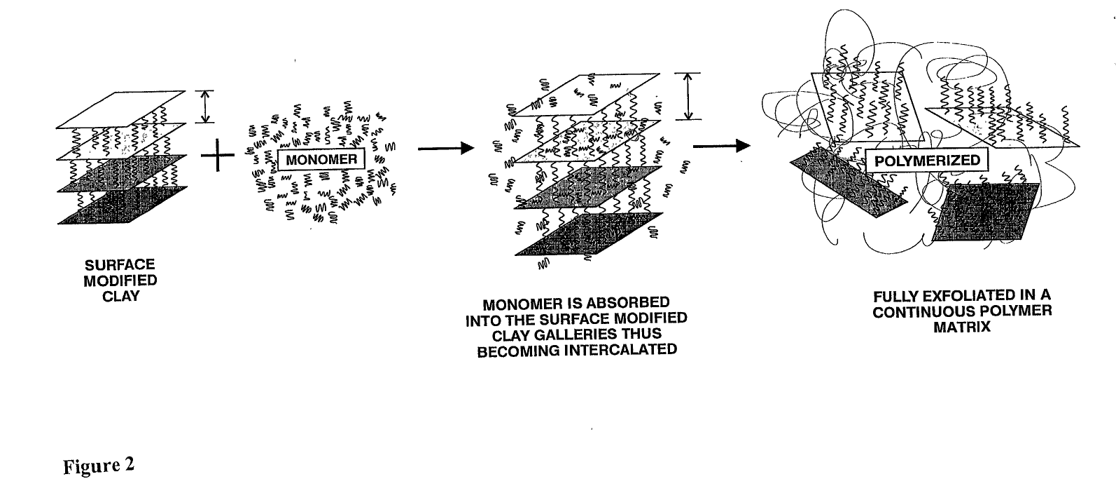

The ion exchange capacity of a clay controls the amount of surface modifier that is able to be bonded to the clay layer. The higher the ion exchange capacity of the clay, the greater amount of surface modification. Montmorillonite clay has an ion exchange capacity between 80 and 140 milliequivalents per 100 grams of clay. Laponite clays have an ion exchange capacity about half that of montmorillonite. The action of this direct correlation between ion exchange capacity and the bifunctional long chain molecule i.e. intercalation which serves to spread apart the galleries increases the inter layer distances to greater than 20 Å. Increasing the distance between each layer reduces inter particle attraction and allows for optimum resin adsorption. This is depicted in FIG. 2.

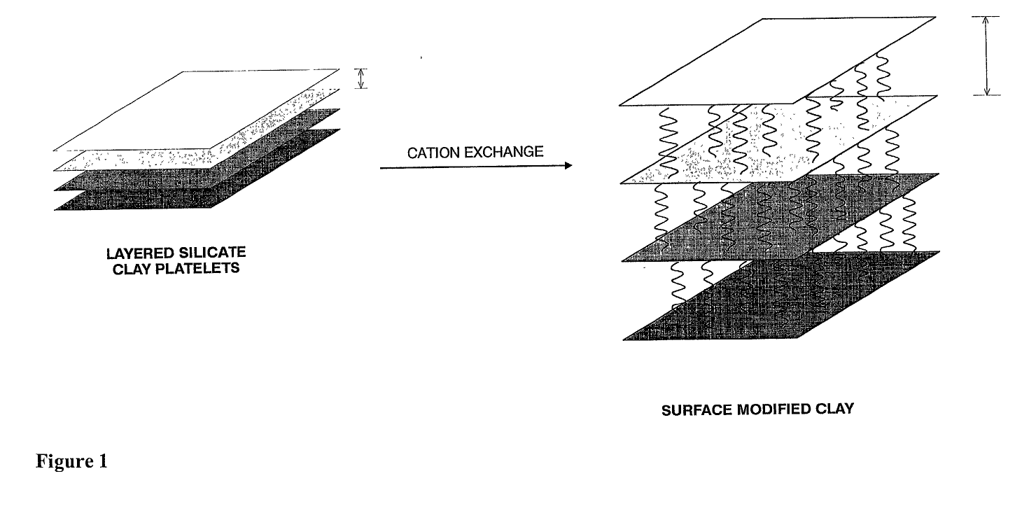

The resins used in dental composites do not optimally swell the clay in its natural state. However, exchanging the hydrated cations with a least one bifunctional organic cation/surface modifier, forces the resin to be adsorbed into the gallery regions and become intercalated. This process is herein referred to as surface treatment and is depicted in FIGS. 1 and 2.

In another preferred embodiment, the resin is a monomer, polymer, oligomer or a combination thereof. Preferably, the monomers, polymers and oligomers are selected from the group consisting of but not limited to acrylic acid monomers, methacrylic acid monomers, acrylate monomers, methacrylate based monomers, styrene monomers, vinyl ether monomers, acrylonitrile monomers, propylene monomers, vinyl acetate monomers, vinyl alcohol monomers, vinyl chloride monomers, vinylidine chloride monomers, butadiene monomers, isobutadiene monomers, isoprene monomers, divinyl benzene and mixtures thereof, polyamides, polyesters, polyolefins, polyimides, polyacrylate, polyurethane, vinyl esters, epoxy based materials, styrene, styrene acrylonitrile, ABS polymers, polysulfones, polyacetals, polycarbonate, polyphenylensulfidies and mixtures thereof, acyrylic oligomers, methacrylic oligomers, styrene oligomers, vinyl ester oligomers, polyester oligomers and mixtures thereof. Most preferably, the resin is a methacrylate based resin. Especially preferred methacrylate based resins include, for example those disclosed in U.S. Pat. Nos. 3,066,112, 3,179,623, 3,194,784, 3,751,399, 3,926,906, and 5,276,068, all of which are herein incorporated by reference in their entirety, and 1,6 hexanediol dimethacrylate, bisphenol “a” dimethacrylate, butyl methacrylate, dimethyl aminoethyl methacrylate, diureathane dimethacrylate, ethoxylated bisphenol “A” dimethacrylate, ethyl methacrylate, hydroxyethyl methacrylate, isobutyl methacrylate, lauryl methacrylate, methyl methacrylate, bisphenol “A” diglycidyl methacrylate, stearyl methacrylate, tetrahydrofufuryl methacrylate, triethylene glycol dimethacrylate, and trimethacrylate.

The surface treatment modifies the hydrophilic silicate to increase the absorptivity of the resin between each layer. The preferable surface treatment allows full intercalation or exfoliation (intercalation refers to the stacking of silicate platelets whereas exfoliation refers to separation of the individual layers into a continuous resin matrix; see FIG. 2). If the optimum surface treatment is not established, not all the galleries are interlayed by layers of monomer, polymer or oligomer, which inhibits even dispersion and greatly limits the nanocomposite properties. In addition, surface treatment with polymerizable functionality chemically bonds the organic matrix to the inorganic nanofiller. By identifying the correct surface modifiers, not only are the nanosized layers intercalated or exfoliated throughout the resin, but the layers are also chemically bonded to the resin via multiple mechanisms. Ultimately the material is strengthened by the intimacy of the interfaces between the organic and the inorganic, which optimizes the load transfer between each phase.

As the polymerization proceeds, the galleries become increasingly congested with resin and the silicate platelets are gradually forced apart until they are separated beyond their inter layer attraction, leading to a well exfoliated nanocomposite. FIG. 2 depicts the action of exfoliation into a continuous resin matrix of monomer, polymer or oligomer-silicate platelets. Small angle x-ray diffraction analysis is used to confirm that silicate platelets are uniformly intercalated or exfoliated throughout the resin matrix. Typical layer spacing of a well exfoliated composite range from 50-200 Å. Transmission electron microscopy (TEM), coupled with a x-ray diffractometer, is a most useful method for measuring the spacing or orientation of these dispersed silicate platelet. Layer spacing of montmorillonite treated with Octadecyl Trimethyl amine increases 36% from a natural state of 25-26 Å to 41 Å after polymerization. Also, Polyoxyethylene Decyloxypropylamine increases spacing of smectite by 22%, from 28-29 Å to that of 37 Å. The larger the spacing range, the more optimum the result.

In a more preferred embodiment, FIG. 2 also depicts the accessibility of the surface treatment to the resin after intercalation or exfoliation. This positioning allows optimal surface treatment thereby fully incorporating the resin for bonding during polymerization. Binding of the resin to the surface modifier allows for a more flexible resin to transfer stress to the stiffer layers.

In another more preferred embodiment, bifunctional coupling agents or silanes are used in combination with the surface modifier to improve physical and mechanical properties and to provide hydrolytic stability by preventing water from penetrating along the silicate/resin interface. In some composites, if the silicate platelets are not bonded to the resin, they can actually weaken the material. Examples of bifunctional coupling agents include, but are not limited to, organo-functional silanes. The bifunctional coupling agent used in dental composites is, preferably, a methacryloxy silane, which co-polymerizes with methacrylate-based resin. The bifunctional compound contains a silicon-functional group that hydrolyses and reacts with active sites on the inorganic surface and an organo-functional group that co-polymerizes with free radical cured resin. Specifically, the silane coupling agent bonds to the edges of the platelets where the necessary hydroxyl groups are present. The platelet edges represent only 1% of the total surface area, which restricts the use of silanation and explains why it is a useful adjunct to the surface modifiers.

Optionally, the nanocomposite of the present invention can also contain a filler, such as for example, a quartz or a glass filler. Other optional materials that can be added to the present invention include, but are not limited to, 2,4-dihydroxy benzophenone, 2,6-di-tert-butyl-4-methylphenol, color pigments, initiators, polymerization accelerators, titanium dioxide, aluminum oxide, fumed silica, photoinitiators, plasticizers, ultra-violet light absorbers and stabilizers, anti-oxidants and other additives well known in the art. To achieve optimum strength and maintain critical handling properties, the nanosized silicate platelet layers will be used in conjunction with a filler. As found in most applications, only 0.05% -90% of the nanosized particles are needed to achieve optimum strength. Preferably, 0.05% -20% of the nanosized particles are needed to achieve optimum strength. Dramatic physical changes to the final material are affected by only a small amount of change in nanofiller loading, nanofiller loading being defined as the percent addition of silicate. Preferably, the amount of nano-sized and existing filler are needed to yield the highest strength without loosing the critical handling properties required.

The ability for the nano-sized particles to uniformly disperse gives the nanocomposite technology a definite advantage over other methods for producing nanocomposites. This method for incorporation of nano-sized particles is unique in that many major property enhancements are realized. It is found that a 0.68% filler loading of a montmorillonite clay modified with Octadecyl Trimethyl amine yielded a 15% increase in compressive strength over that of current dental composite material.

Having generally described the invention, a more complete understanding can be obtained with reference to certain specific examples, which are included for purposes of illustration only. It should be understood that the invention is not limited to the specific details of the Examples. Starting materials may be obtained from commercial sources, prepared from commercially available compounds, or preferred using well known synthetic methods.

### EXAMPLE 1

Self-cure Dental Tooth Filling Composite. Two pastes (a base paste and a catalyst paste) are mixed in a 1:1 (w/w) ratio to form a peroxide/amine intitatied polymerized tooth filling composite.

Base Paste

| | % |
|---|---|
| Chemical | Range |
| Proprietary Blend of | 10-75 |
| Methacrylate | |
| Monomers #01916O3 | |
| N,N-Bis(2,Hydroxyethyl)-p- | 0-3 |
| Toludine | |
| 2,4 Dihydroxy Benzophenone | 0-3 |
| Multi micron size Barium Glass | 5-95 |
| Filler | |
| color pigments | 0-3 |
| Titanium Dioxide | 0-3 |
| Fumed Silica | 0-10 |
| Montmorillonite clay Treated | 1-20 |
| with Octadecyl Trimethyl Amine | |
| Total | 100 |

Catalyst Paste

| | % |
|---|---|
| Chemical | Range |
| Proprietary Blend of | 10-75 |
| Methacrylate | |
| Monomers #0191604 | |
| 2,6-Di,Tert,Butyl-4- | 0-3 |
| Methylphenol | |
| Benzoyl Peroxide | 0-3 |
| Micron sized quartz glass filler | 5-95 |
| Aluminum oxide | 0-10 |
| Fumed silica | 0-10 |
| Montmorillonite clay Treated | 1-20 |
| with Octadecyl Trimethyl Amine | |
| Total | 100 |

The disclosures in this application of all articles and references, including patents, are incorporated herein by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A nanocomposite for use in dental applications, the nanocomposite comprising:
a plurality of silicate platelets;
one or more regions spacing the plurality of silicate platelets from each other;
at least one surface modifier ion-exchanged to each of the plurality of silicate platelets; and
a dentally compatible resin absorbed into the regions spacing the plurality of silicate platelets, the platelets and resin forming an intercalated or exfoliated structure.

2. A nanocomposite intermediate for use in dental applications, the nanocomposite intermediate comprising:
a plurality of silicate platelets;
one or more regions spacing the plurality of silicate platelets from each other;
at least one surface modifier ion-exchanged to each of the plurality of silicate platelets; and
a dentally compatible resin absorbed into the regions spacing the plurality of silicate platelets.

3. A nanocomposite according to claim 1 prepared by the process comprising:
providing a plurality of silicate platelets having one or more regions spacing the plurality of silicate platelets from each other;
ion-exchanging at least one surface modifier to the surface of each of the plurality of silicate platelets;
absorbing a dentally compatible resin into the regions spacing the plurality of silicate platelets; and
modifying the dentally compatible resin such that an intercalated or exfoliated structure is created.

4. A nanocomposite intermediate according to claim 2 prepared by the process comprising:
providing a plurality of silicate platelets having one or more regions spacing the plurality of silicate platelets-from each other;
ion-exchanging at least one surface modifier to the surface of each of the plurality of silicate platelets; and
absorbing a dentally compatible resin into the regions spacing the plurality of silicate platelets.

5. A method of making a nanocomposite according to claim 1 comprising the steps of:
providing a plurality of silicate platelets having one or more regions spacing the plurality of silicate platelets from each other;
ion-exchanging at least one surface modifier to the surface of each of the plurality of silicate platelets;
absorbing a dentally compatible resin into the regions spacing the plurality of silicate platelets; and
modifying the dentally compatible resin such that an exfoliated structure is created.

6. A method of making a nanocomposite intermediate according to claim 2 comprising the steps of:
providing a plurality of silicate platelets having one or more regions spacing the plurality of silicate platelets;
ion-exchanging at least one surface modifier to each of the plurality of silicate platelets; and
absorbing a dentally compatible resin into the regions spacing the plurality of silicate platelets.

7. A method of using a solid nanocomposite for dental applications, the method comprising the steps of:
providing a solid nanocomposite, the nanocomposite comprising: a plurality of silicate platelets; one or more regions spacing the plurality of silicate platelets from each other; at least one surface modifier ion-exchanged to each of the plurality of silicate platelets; a dentally compatible resin is absorbed into the regions spacing the plurality of silicate platelets, and the platelets and resin forming an intercalated or exfoliated structure.

8. The nanocomposite of claim 1 wherein said plurality of silicate platelets is selected from the group consisting of smectite clay, vermiculite, halloysite, a mixed layered clay, a mica or sericite.

9. The nanocomposite of claim 8 wherein said smectite clay is selected from the group consisting of montmorillonite, laponite, saponite, beidellite, nontronite, hectorite, swellable mica based mineral, stevensite or any synthetic analog thereof.

10. The nanocomposite of claim 8 wherein said silicate platelets are used in conjunction with an additive.

11. The nanocomposite of claim 10 wherein said additive is selected from the group consisting of quartz filler, glass filler, 2,4-dihydroxy benzophenone, 2,6-di-tert-butyl-4-methylphenol, color pigments, initiators, polymerization accelerators, titanium dioxide, aluminum oxide, fumed silica, photoinitiators, plasticizers, ultra-violet light absorbers and stabilizers, and anti-oxidants.

12. The nanocomposite according to claim 1 wherein said gallery region spacing is in the range of 3.5 Å-200 Å.

13. The nanocomposite according to claim 1 wherein the at least one surface modifier is an organic cation.

14. The nanocomposite according to claim 13 wherein said organic cation is selected from the group consisting of Bis(2-Hydroxyethyl) methyl tallow quaternary ammonium ion, dimethyl-2-ethyl hexyl hydrogenated tallow quaternary ammonium ion, methyl dihydroxyethyl hydrogenated tallow ammonium, aminododecanoic acid, polyoxyethylene decyloxypropylamine, and octadecyl trimethyl amine.

15. The nanocomposite according to claim 13 wherein the at least one surface modifier is used in combination with bifunctional coupling agents or silanes.

16. The nanocomposite according to claim 15 wherein said bifunctional coupling agent is a methacryloxy silane.

17. The nanocomposite according to claim 1 wherein said resin is a monomer, polymer, oligomer or a combination of the like.

18. The nanocomposite of claim 17 wherein said monomer is selected from the group consisting of acrylic acid monomers, methacrylic acid monomers, acrylate monomers, methacrylate based monomers, styrene monomers, vinyl ether monomers, acrylonitrile monomers, propylene monomers, vinyl acetate monomers, vinyl alcohol monomers, vinyl chloride monomers, vinylidine chloride monomers, butadiene monomers, isobutadiene monomers, isoprene monomers, divinyl benzene and mixtures thereof.

19. The nanocomposite of claim 17 wherein said polymer is selected from the group consisting of polyamides, polyesters, polyolefins, polyimides, polyacrylate, polyurethane, vinyl esters, epoxy based materials, styrene, styrene acrylonitrile, ABS polymers, polysulfones, polyacetals, polycarbonate, polyphenylensulfidies and mixtures thereof.

20. The nanocomposite of claim 17 wherein said oligomer is selected from a group consisting of acyrylic oligomers, methacrylic oligomers, styrene oligomers, vinyl ester oligomers, polyester oligomers and mixtures thereof.

21. A method of using resin-silicate layered nanocomposite for dental applications, the method comprising:
providing a resin-silicate layered nanocomposite, the nanocomposite comprising: a plurality of silicate platelets; one or more gallery regions spacing the silicate platelets; at least one surface modifier ion-exchanged to each silicate platelet; an intercalated structure such that resin is absorbed into the gallery regions spacing the silicate platelets; and an exfoliated structure lying in a continuous resin matrix such that a solid nanocomposite is formed; and
using the resin-silicate layered nanocomposite in a dental application.

22. The method of claim 21, wherein the dental application includes use in dental composite restorative materials.

23. The method of claim 21, wherein the dental composite restorative materials are selected from the group consisting of sealants, core materials, adhesives, bonding agents, veneering materials, cements, dentures, inlays, microfill composites, flowable composites, compomers, anterior composites, posterior composites, resin modified glass ionomes, and condensable composites.

24. The method of claim 23, wherein the dental composite restorative materials can be light cured, self cured or combination thereof.

25. The method of claim 21, wherein the dental application includes use in dental appliances, orthodontic devices and appliances, bite plate appliances, denture base resins, temporary and permanent crowns and bridges.

26. The method of claim 21, wherein the dental application includes use in orthopedic appliances, acrylic prosthesis, bone cements, and adhesives.
